(19) United States
(12) Patent Application Publication
(10) Pub. No.: US 2021/0045495 A1
(43) Pub. Date: Feb. 18, 2021

(54) ORTHOTIC DEVICE FOR HAPTIC TERRAIN FEEDBACK AND CONTROL

(71) Applicant: University of Utah Research Foundation, Salt Lake City, UT (US)

(72) Inventors: Mark Minor, Salt Lake City, UT (US); Yue Wang, Sunnyvale, CA (US); Andrew Merryweather, Salt Lake City, UT (US)

(21) Appl. No.: 16/993,682

(22) Filed: Aug. 14, 2020

Related U.S. Application Data
(60) Provisional application No. 62/886,802, filed on Aug. 14, 2019.

(51) Int. Cl. A43B 13/20; G06F 3/01; A43B 3/00; A61B 5/11

(52) U.S. Cl. CPC A43B 13/203; G06F 3/011; A61B 2562/0247; A61B 5/112; A61B 2562/0219; A43B 3/0005

(57) ABSTRACT

An orthotic device for haptic terrain feedback and control includes a plate having a mechatronic unit, a bladder structure secured to the plate, the bladder structure having one or more bladder cells, and a passageway extending between the bladder cell and an environment outside the orthotic device. The orthotic device is configured to provide haptic feedback to a user by way of selective inflation and deflation of the one or more bladder cells.

### CROSS-REFERENCE TO RELATED APPLICATIONS

This application claims priority to and the benefit of U.S. Provisional Patent Application No. 62/886,802, filed Aug. 14, 2019 and titled “Orthotic Device for Haptic Terrain Feedback and Control,” the entirety of which is incorporated herein by this reference.

### STATEMENT REGARDING FEDERALLY SPONSORED RESEARCH

This invention was made with government support under grant no. 1162617 awarded by the National Science Foundation. The government has certain rights in the invention.

### BACKGROUND

### 1. The Field of the Invention

The present disclosure relates generally to systems, methods, and apparatus for terrain-enabled Virtual Reality (VR) based gait therapy. More specifically, the present disclosure relates to an orthotic device for haptic terrain control.

### 2. Background and Relevant Art

Haptic terrain enabled systems can be used for gait rehabilitation, training, and entertainment purposes. For example, haptic terrain displays can be used by patients during physical therapy or other gait training applications to compensate for uneven or rough terrain to reduce instances of imbalance and falling while walking. Haptic terrain enabled systems can also be used in conjunction with VR and augmented reality-based environments to produce haptic feedback of virtual terrain features while walking on a flat surface. Similar systems used in conjunction with VR-based environments also have potential to improve VR-based gaming and entertainment experiences.

Virtual environments have become increasingly popular in recent years, with the most common and immersive virtual environments including systems where a user can walk in a graphical virtual world rendered by projectors. In such virtual worlds, a treadmill is usually introduced to provide walking capability. However, due to the planar workspace of the treadmill, the haptic display of the virtual terrain is very limited. As such, most current terrain display and locomotion interfaces are limited to rendering simple/gross terrain features, such as slopes and stairs. These gross terrain features are far from the realistic features of textures, small bumps, rocks, and other fine terrain features encountered in the real world.

There have been developments in the area of orthotic devices that provide haptic terrain feedback within a virtual world, but the majority of such devices focus on vibrotactile feedback, which is not effective alone for rendering fine surface features, in particular for small features such as small rocks, gravel, or cobblestone streets, for example. Other orthotic devices for virtual terrain rendering are limited in resolution and are complicated to use and manufacture. For example, current devices require significant power sources, bulky electronics, and other complicated mechanical and electromechanical components. As a result, these orthotic devices are large and heavy, such that the use thereof results in the impedance of a user's natural walking motion. Such impedance can lead to injury or can exacerbate injury, rather than provide entertainment and/or rehabilitation.

In addition, current devices developed for haptic terrain feedback are too expensive for general consumers, relatively expensive to manufacture, and not easily scalable. Accordingly, there are a number of problems in the prior art that can be addressed.

### BRIEF SUMMARY

Embodiments described in the present disclosure solve one or more problems in the art through systems and methods related to terrain-enabled Virtual Reality based gait therapy. More specifically, the present disclosure relates to an orthotic device for haptic terrain control. In one embodiment of the present disclosure, an orthotic device for haptic terrain feedback and control includes a plate having a mechatronic unit, a bladder structure secured to the plate, the bladder structure having a bladder cell, and a passageway extending between the bladder cell and an environment outside the orthotic device.

In one embodiment of the present disclosure, an orthotic device for haptic terrain feedback and control includes a bladder structure having a plurality of bladder cells, and a mechatronic plate having one or more mechatronic units disposed therein, each mechatronic unit of the one or more mechatronic units comprising a valve. In such an embodiment, the valves are configured to be selectively opened and closed to allow a gas to flow in and out of at least one of the plurality of bladder cells.

In one embodiment of the present disclosure, an orthotic device for terrain control includes a bladder structure and a plate. The bladder structure includes a plurality of bladder cells and one or more sidewalls at least partially defining the plurality of bladder cells. The plate is secured to the bladder structure. The plate includes a mechatronic unit, a port, and a passageway extending through the plate between at least one of the plurality of bladder cells and the port.

Embodiments described herein can provide a number of benefits. For example, an orthotic device can provide both kinesthetic cues (e.g., foot, limb, and body pose changes resulting from altering the shape of the bladder structure) and cutaneous cues (e.g., forces directed to the sole of the foot as a result of varying shape of the bladder structure and changing internal pressure). Use of an orthotic device offers the ability to replicate training scenarios in a safe and controlled environment, which may be beneficial for those in need of physical therapy and/or those with walking impediments such as Parkinson's disease (PD), spinal cord injuries, post-stroke complications, and the like.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and should not therefore be considered to be limiting of its scope, the invention will be described and explained with additional specificity and detail through the use of the accompanying drawings in which:

FIG. 1 illustrates a perspective view of a user wearing an orthotic device, including a sole and an upper, according to the present disclosure;

FIG. 2 illustrates six cross-sectional views (a)-(f) of the orthotic device illustrated in FIG. 1, taken along plane 2-2 indicated in FIG. 1, in various haptic terrain control configurations based on a variety of terrain features, according to the present disclosure;

FIG. 3A illustrates a bottom perspective view of an embodiment of an orthotic device, including a plate secured to a lower bladder structure, according to the present disclosure;

FIG. 3B illustrates an exploded view of an embodiment of an orthotic device, including a plate integrated with mechatronic units and a bladder structure, according to the present disclosure;

FIG. 4A illustrates top views of various embodiments of bladder structures, according to the present disclosure;

FIG. 4B illustrates a perspective view of an embodiment of a bladder having rounded individual cellular bladders, according to the present disclosure;

FIG. 5A illustrates a schematic view of an embodiment of a mechatronic unit of a plate, according to the present disclosure;

FIG. 5B illustrates a schematic view of a plurality of mechatronic units connected together within a plate of an orthotic device, according to the present disclosure;

FIG. 6 illustrates a cross-sectional view of an embodiment of an orthotic device, taken along line 6-6 indicated in FIG. 3A, including various mechatronic units integrated into a plate that interface with cellular bladders, according to the present disclosure;

FIG. 7A illustrates a perspective view of an embodiment of a valve used in a plate of an orthotic device, according to the present disclosure;

FIG. 7B illustrates a cross-sectional view of the valve illustrated in FIG. 7A, taken along line 7B-7B indicated in FIG. 7A, according to the present disclosure;

FIG. 8 illustrates a perspective view of a mold used to form a plate of an orthotic device, according to the present disclosure; and

FIG. 9 illustrates a user wearing an embodiment of an orthotic device within a VR-based environment.

### DETAILED DESCRIPTION

### Introduction

Embodiments described in the present disclosure solve one or more problems in the art with systems, methods, and devices related to terrain-enabled VR-based gait therapy. More specifically, the present disclosure relates to an orthotic device for haptic terrain control. Embodiments described herein solve one or more of the problems in the prior art noted above. For example, embodiments of the present disclosure provide orthotic devices capable of producing high definition haptic feedback of fine terrain features, either in real world use (e.g., during rehabilitative therapy or a training exercise) or a VR-based environment, including augmented reality (AR) environments.

Orthotic devices of the present disclosure are comparatively inexpensive and simple to manufacture. In addition, orthotic devices of the present disclosure are relatively lightweight and small such that the user's natural gait is not impeded during use. The orthotic devices of the present disclosure are wearable and portable such that a user can wear the orthotic devices as one would wear any other shoe as part of everyday life.

### Overview of Exemplary Orthotic Devices

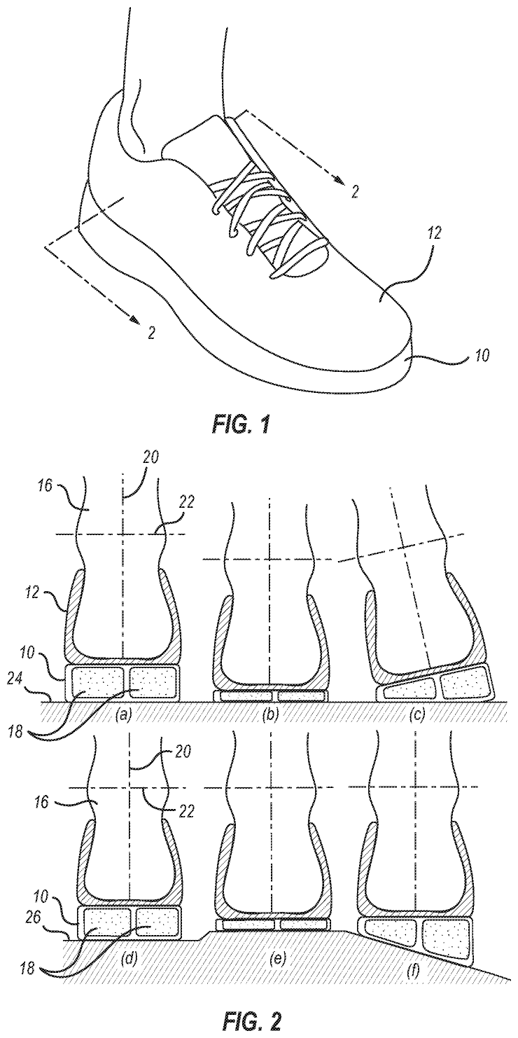

Turning now to the figures, FIG. 1 illustrates an embodiment of an orthotic device 10 secured to a shoe upper 12 worn by a user during rehabilitation, training, or within a VR-based environment, including AR-based environments. In general, orthotic device 10 is secured to upper 12. Upper 12 can be formed as any common shoe upper currently known in the art configured to be attached to a sole. In this way, users can wear orthotic device 10 and enjoy the aesthetic appearance of a common athletic shoe, dress shoe, sandal, hiking shoe, or other commonly worn shoe. In at least one embodiment, upper 12 is secured to orthotic device 10, which acts as a sole, via adhesion, molding, stitching, other common shoe-to-sole securement methods known in the art, or a combination thereof.

In at least one embodiment, orthotic device 10 is removably secured to upper 12 such that orthotic device 10 can be used with multiple uppers. In such an embodiment, a user may have the option to use a variety of different embodiments of orthotic devices 10 with the same upper 12. In addition, in such an embodiment, a user may have the option to use a variety of different uppers 12 with the same embodiment of orthotic device 10.

FIG. 2 illustrates six cross-sectional views (a)-(f) of the orthotic device 10 illustrated in FIG. 1, taken along plane 2-2 indicated in FIG. 1, in various haptic terrain control configurations based on a variety of terrain features. Each view (a)-(c) illustrates a user's lower leg and ankle 16 disposed within an orthotic device 10, including upper 12 and sole, with orthotic device 10 either causing or compensating for terrain features resulting in foot inversion, eversion, dorsiflexion and plantar flexion. In general, sole 10 includes a plurality of bladder cells 18 filled with air or other gas.

A longitudinal axis 20 is disposed through lower leg and ankle 16 and a lateral axis 22 is disposed across lower leg and ankle 16. In a properly aligned, stable position, vertical axis 20 is disposed vertically or substantially vertical and lateral axis 22 is disposed horizontally or substantially horizontal relative to ground/floor surface 24. Cross-sectional views (a)-(c) of FIG. 2 illustrate orthotic device 10 being worn on a flat surface 24.

As shown in cross-sectional views (a)-(c) of FIG. 2, in at least one embodiment, bladder cells 18 of sole 12 can be deflated and inflated to various degrees and potentially differential volumes to alter the position of lower leg and ankle 16, including the relative positions and angles of vertical axis 20 and lateral axis 22, relative to surface 24. It will also be noted that alterations in the position of the user's lower leg and ankle 16 results in alterations in the entire body position of the user during walking or standing. For example, as shown in cross-sectional view (a) of FIG. 2, both a left and a right bladder cell 18 are inflated with air equally such that lateral axis 22 is horizontal relative to surface 24 and vertical axis 20 is vertical relative to surface 24. Cross-sectional view (b) shows both the left and the right bladder cell 18 deflated relative to bladder cells 18 shown in cross-sectional view (a) such that vertical axis 20 is still vertical and lateral axis 22 is still horizontal, but lateral axis 22 is closer to surface 24.

Cross-sectional view (c) of FIG. 2 illustrates an alternative haptic terrain control configuration showing left and right bladder cells 18 inflated to different degrees such that lateral axis 22 is no longer horizontal to surface 24 and vertical axis 20 is no longer vertical relative to surface 24, resulting in foot eversion. A more detailed description regarding the precise construction, configuration, and functionality of various embodiments of the orthotic device 10, including the inflation and deflation of various bladder cells 18, the number and position of bladder cells 18, and operation of the orthotic device 10, will be given with reference to subsequent figures. FIG. 2 merely illustrates a number of non-limiting examples of an embodiment of orthotic device 10 altering the position of lower leg and ankle 16 of a user relative to surface 24.

More specifically, cross-sectional views (a)-(c) illustrate how an embodiment of orthotic device 10 can provide haptic terrain feedback of various terrain features and slopes to a user walking on flat ground. Such capabilities can be used to produce haptic feedback of terrain features rendered in a VR-based or AR-based environment. That is, using a treadmill or other planar walking surface which is void of varying terrain features, for example within a VR-based environment, embodiments of orthotic device 10 are able to manipulate lower leg and ankle 16 positions to mimic various terrain features within a VR-based environment and provide haptic feedback accordingly.

Cross-sectional views (d)-(f) of FIG. 2, on the other hand, illustrate how orthotic device 10 can compensate for various terrain features to keep lateral axis 22 level across various surface features and to keep vertical axis 20 vertically disposed. Surface 26 includes raised and sloped portions shown in cross-sectional views (e) and (f), respectively. Again, bladder cells 18 can be variably inflated and deflated to compensate for the raised and sloped portions of surface 26 to maintain lower leg and ankle 16 in a stable position. In such scenarios, orthotic device 10 can stabilize the lower leg and ankle 16 of a user who may otherwise become imbalanced on such an uneven surface 16 or surfaces that include other unpredictable terrain features or that would otherwise cause foot movement such as inversion, eversion, dorsiflexion, and/or plantar flexion. As noted above, a more detailed description of various configurations and functionalities of orthotic device 10, including the inflation and deflation of bladder cells 18, will be given with reference to subsequent figures.

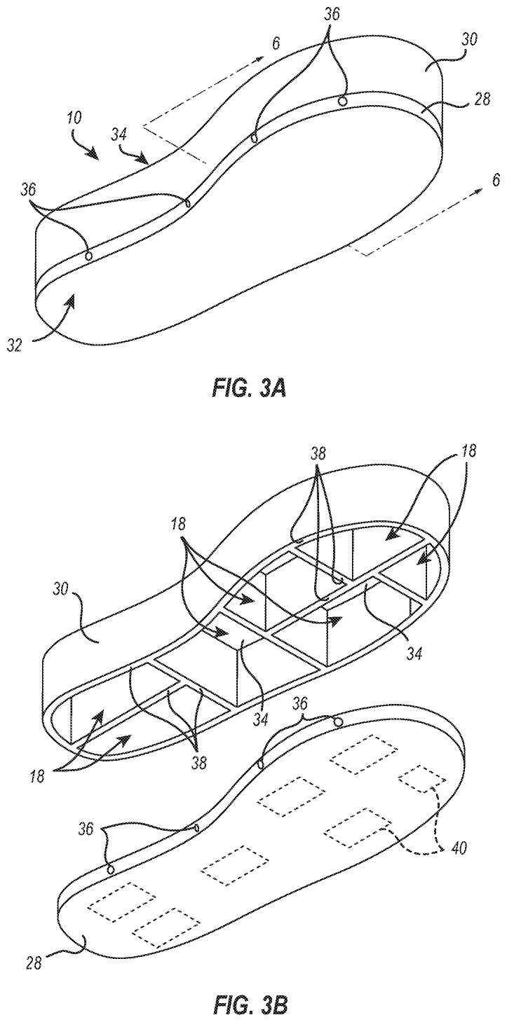

With reference to FIG. 3A, embodiments of orthotic devices 10 described herein generally include a plate 28 disposed on the bottom 32 of a bladder structure 30. FIG. 3A illustrates a bottom perspective view of an embodiment of an orthotic device 10, including plate 28 secured to bladder structure 30. In the illustrated embodiment of FIG. 3A, plate 28 is secured to bottom 32 of bladder structure 30 such that during use, plate 28 contacts the ground. In at least one embodiment, plate 28 may include tread features to promote grip and friction between plate 28 and the ground during use. Such tread features may be similar to tread features commonly found on shoes as is known in the art.

In embodiments where plate 28 is disposed on the bottom of bladder structure 30, an upper surface 34 of bladder structure 30 is closed to provide a surface on which a shoe upper, such as upper 12 illustrated in FIG. 1, can be attached. Closed upper surface 32 of bladder structure 30 also may provide a surface to support a user's foot and define a closed upper boundary of each bladder cell 18 (not illustrated in FIG. 3A). In such an embodiment, the upper surface of plate 28 defines a closed lower boundary of each bladder cell 18.

Alternatively, in at least one embodiment, plate 28 is formed as upper surface 34 of bladder structure 30 such that bladder structure 30 is in direct contact with the ground during use and plate 28 forms an upper closed boundary for each bladder cell 18. In such an embodiment, bladder structure 30 may include tread features noted above and plate 28, disposed on top of bladder structure 30, serves to support the foot of the user and can be used to provide an upper surface to form to shoe upper 12 as shown in FIG. 1.

In at least one embodiment, such as the embodiment illustrated in FIG. 3A, where plate 28 is disposed below bladder structure 30 to form a lower surface of orthotic device 10, plate 28 may be stiffer than bladder structure 30 (e.g., formed of a material with a higher modulus of elasticity) and/or harder than the bladder structure 30 (e.g., have a higher Shore A hardness). Plate 28 may also be more durable, since plate 28 interacts with the ground during use and preferably withstands repeated use over hard, rough terrain. Likewise, bladder structure 30 is formed of compliant materials that allow internal bladder cells 18 to expand, contract, and change shape during use such that a user, can feel the changes in volume and shape of internal bladder cells 18 interacting directly with the user's foot during use.

In addition, in at least one embodiment, ports 36 extend through plate 28 and selectively communicate with internal bladder cells 18 inside bladder structure 30. To further clarify the internal bladder cells 18 within bladder structure 20 referenced above, FIG. 3B illustrates an exploded view of an embodiment of an orthotic device 10, including plate 28 and bladder structure 30. As shown, sidewalls 38 separate an internal volume of bladder structure 30 into distinct bladder cells 18 within bladder structure 30. Each bladder cell 18 is defined by two or more sidewalls 38, upper surface 34, and plate 28.

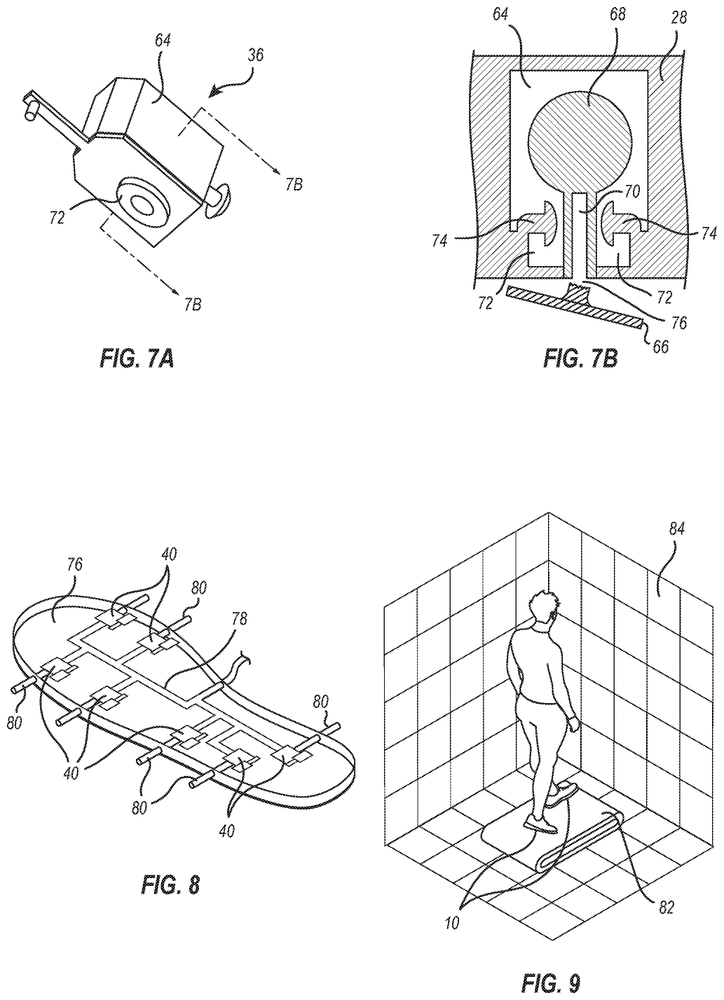

A gas, such as air, occupies each bladder cell 18 and may controllably enter and exit each bladder cell 18 during use through ports 36, which extend through plate 28 and communicate with each bladder cell 18. Each port 36 is selectively opened and closed via a valve mechanism, which will be discussed in more detail with reference to subsequent figures, including FIGS. 6, 7A, and 7B. However, in general, plate 28 is equipped with mechatronic units 40, which in at least one embodiment, are embedded within or on plate 28. Each mechatronic unit 40, which is generally associated with an individual bladder cell 18, includes valve controls that open and close valves associated with each port 36 to selectively allow gases to enter into each bladder cell 18 from outside orthotic device 10 and exit each bladder cell 18 into the environment outside orthotic device 10. Again, more detail regarding the valve mechanisms and mechatronic units 40 will be given hereafter.

First, with reference to bladder cells 18 shown in FIG. 3B, each bladder cell 18 can selectively expand and contract during use to replicate small bumps and terrain features, such as illustrated in FIG. 2, which may be rendered in a virtual environment. With each step of a user, each port 36 selectively allows gases to pass into or out of each bladder cell 18 such that each bladder cell 18 can individually expand or contract as needed to replicate a virtual environment under a user's foot or compensate for terrain features in the real world to assist in keeping a user's foot level.

In at least one embodiment, the passing of gases in and out of each bladder cell is passive, such that a user's weight forces air out of a bladder cell 18 with an open port 36 when the user steps on the ground. In such a passive scenario, sidewalls 18 elastically rebound between steps during a swinging portion of a user's gait when the user is not stepping on the ground to return each bladder cell 18 that has contracted back to its resting volume. In this way, a controller communicates with mechatronic units 40 to selectively open ports 36 to allow bladder cells 18 corresponding to certain terrain features to remain expanded or to contract to various degrees, thus imitating virtual terrain features or compensating for terrain features in the real world and providing corresponding tactile feedback to the user whose foot is interacting with bladder structure 30.

In such a passive configuration, which utilizes the user's weight to force air out of individual bladders through plate 28 with open ports 36, sidewalls 38 comprise materials that are flexible enough to contract rapidly during contraction (e.g., rapidly enough to contract completely or substantially during a typical step) but stiff enough to rebound back to a resting state or volume during the lifting and swinging portions of the step, creating a pressure vessel of each bladder cell 18. In addition, sidewall 38 materials are preferably materials that maintain sufficient elasticity over repeated uses and those that do not allow bladder cells 18 to balloon outward too far during use, which can cause total collapse of bladder cells 18 or unpredictable bending of sidewalls 38.

Materials used for bladder structure 30 and corresponding sidewalls 38 can be any material that meets the functional requirements noted above, including sufficient flexibility, elasticity, and durability. In at least one embodiment, such materials include rubbers, plastics, foams, urethanes, other polymers and fabric composite materials including fabric-polymer composites (e.g., rubbers or other polymers with embedded fabrics), other fiber/fabric reinforced polymer materials, or combinations thereof. In at least one embodiment, such materials of the bladder structure 30 and sidewalls 38 thereof include blended materials such as polymer blends and other synthetic blends. For example, in at least one embodiment, the bladder structure 30 and sidewalls 38 thereof include a cotton-polymer blend.

Bladder wall materials should be compliant, recover their original shape quickly, and be sufficiently durable to withstand repeated flexing. Examples of specific materials used in the bladders include silicone rubber and reinforced rubber composites. Materials that could be embedded in the rubber could include cotton fabric, cotton-polyester blends, and rayon, to name a few. Composite materials improve durability and strength of bladder materials, which is important for maintaining bladder integrity for extended usage. Composite materials such as cotton-polyester blend that provide excellent bonding and strength improvements are important for improving bladder rigidity and better displaying stiffer surfaces. Stretchy composite materials such as polyether-polyurea copolymer (i.e., spandex) provide improved durability while allowing bladder walls to deform more compliantly to better display soft surfaces.

In one presently preferred embodiment, the orthotic device 10 includes a bladder structure 30 with bladder wall materials formed from a rubber material with embedded fabric. Such orthotic devices have been found to last for over 200,000 cycles of simulated heel strikes without suffering catastrophic failures.

Mechatronic plate materials should be flexible, durable, and should seal well around components. Examples of materials used in the mechatronic plate include silicone rubber, urethane foam, and combinations therein, to name a few. Foam materials are light, but less elastic and less durable. Silicone rubber materials are more durable and well suited to forming sealed structures.

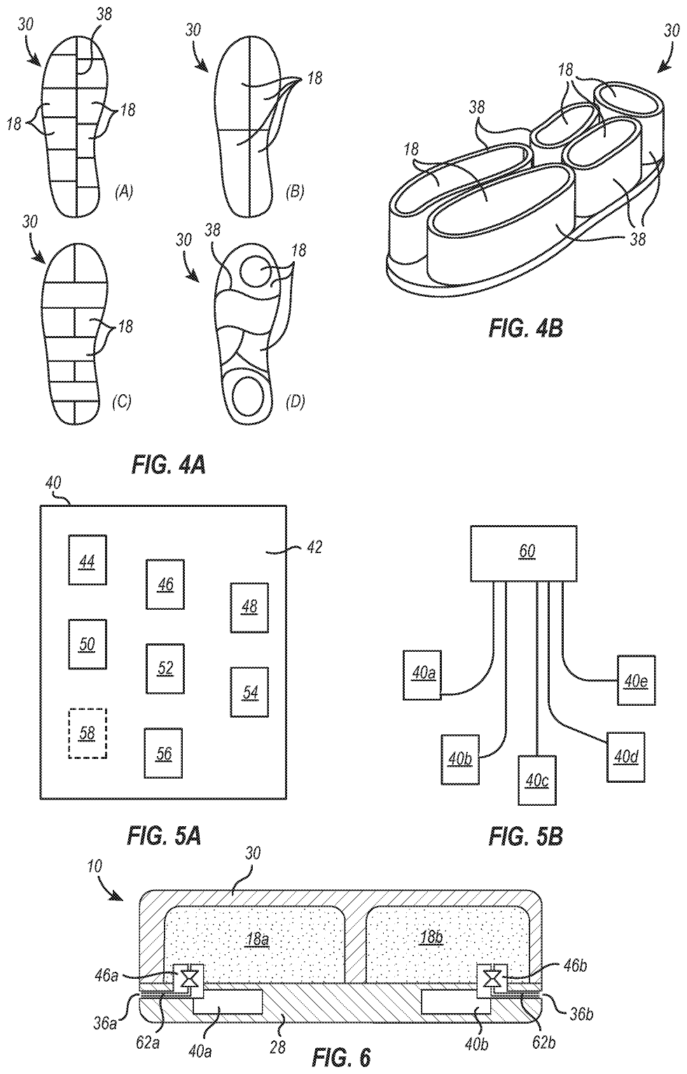

FIG. 4A illustrates top views of various embodiments of bladder structures 30, according to the present disclosure, which utilize different quantities, shapes, sizes and arrangements of bladder cells 18. For example, first bladder structure (A) of FIG. 4A includes twelve individual bladder cells 18 arranged on either side of a central sidewall 38. Alternatively, as shown in second bladder structure (B) of FIG. 4A, bladder structure 30 only includes four bladder cells 18.

Third bladder structure (C) of FIG. 4A includes eleven bladder cells 18, some of which extend entirely laterally across bladder structure 30 and some that do not. Alternatively, as shown in third bladder structure (D) of FIG. 4A, bladder structure 30 includes a variety of bladder cells 18 of various irregular shapes and arrangement. For example, some bladder cells 18 can be circular or have curvilinear sidewalls 38 therebetween. Also, one or more bladder cells 18 may be entirely encompassed within another bladder cell 18.

The number of bladder cells 18 relates to the resolution of orthotic device 10. “Resolution” as used herein refers to the size of the terrain features the orthotic device 10 is able to reproduce for haptic feedback to the user. The smaller the feature which the orthotic device 10 is able to reproduce, the higher the resolution. In general, the higher the number of bladder cells 18 within bladder structure 30, the higher the haptic resolution of orthotic device 10 will be to render finer and finer terrain features.

For example, comparing bladder structure (A) and (B) of FIG. 4A is illustrative of two different haptic resolutions. Bladder structure (A) includes twelve bladder cells 18 and bladder structure (B) includes four. The resolution of the bladder structures 30 is limited to the size of the smallest bladder cell 18 of the particular bladder structure 30. Thus, second bladder structure (B) of FIG. 4A is arranged to inflate or deflate about ¼, at the least, of bladder structure 30 in response to a virtual rock or other terrain feature, even if that rock or terrain feature is only 1/10 the area of bladder structure 30 as seen from the top view in FIG. 4B.

In contrast, because first bladder structure (A) of FIG. 4B includes twelve bladder cells 18 within the same area of bladder structure 30, manipulating a single bladder cell 18 by expansion or contraction results in haptic feedback of a rock or other terrain feature that is as small as 1/12 of the area of bladder structure 30 as seen from the top view of FIG. 4B. Thus, the resolution of first bladder structure (A) is greater than the resolution of second bladder structure (B).

As such, the resolution of orthotic devices 10 described herein is related to the number of bladder cells 18 within the bladder structure 30. In addition, the arrangement of bladder cells 18 and thicknesses of sidewalls 38 may also affect haptic resolution. In any case, the number, arrangement, size, and shape of bladder cells 18 and sidewalls 38 can be customized to meet various resolution needs or other desired performance criteria during manufacturing, such as cost, complexity, durability, and so forth. Effective results have been found when the orthotic device 10 includes at least 4 bladder cells 18 and up to about 10, 12, 14, 16, 18, or 20 bladder cells 18. The upper limit to bladder cells 18 can be higher in certain applications, though such higher levels typically involve diminishing returns in actual user-perceived effects.

FIG. 4B illustrates a perspective view of an embodiment of a bladder structure 30 having a plurality of bladder cells 18, with each bladder cell having a separate sidewall 38 forming separate bladder cells 18. In such an embodiment, at least some of the sidewalls 38 form a boundary of a single bladder cell 18. In at least one embodiment, one or more separate bladder cells 18 are formed having rounded shapes via curvilinear sidewalls 38 such that sidewalls 38 do not form abrupt angles or T-junctions between bladder cells 18. Embodiment with reduced numbers of abrupt sidewall 38 angles and/or T-junctions or may prevent stress concentrations within sidewalls 38, which is beneficial because such stress concentrations can lead to material failure over time with repeated use.

Advantageously, such an embodiment as shown in FIG. 4B prevents unexpected or uneven buckling of sidewalls 38 that can occur between bladder cells 18 that share a common sidewall 38 but are independently expanded or contracted to different volumes during use. Rather, in the illustrated embodiment, each bladder cell 18 is separate from one another so that each bladder cell 18 can expand and contract independently without affecting adjacent bladder cells 18 during use.

### Exemplary Mechatronic Units

FIG. 5A illustrates a schematic view of an embodiment of a mechatronic unit 40 which may be associated with a plate 28, according to the present disclosure. As noted above with reference to FIG. 3B, plate 28 may include one or more mechatronic units 40 embedded therein, with each unit 40 being associated with a bladder cell 18. Each mechatronic unit 40 controls, among other things, the inflow and outflow of gas within each bladder cell 18 through a corresponding port 36. In addition, each mechatronic unit 40 may include a number of components that enable orthotic devices 10 described herein to track motion, monitor foot and ankle position, control valves or other components, and thus accurately render terrain features through bladder structure 30.

For example, in at least one embodiment, mechatronic unit 40, as shown in FIG. 5A, includes at least one or a combination of the following components: circuit board 42, processor 44, valve 46, range finder 48, pressure sensor 50, accelerometer 52, power source 54, and/or gyroscope 56. In at least one embodiment, mechatronic unit 40 can also include other components 58. In at least one embodiment, using the various components noted above, mechatronic unit 40 is able to receive input from a VR-based or AR-based environment or a real-world environment and cause bladder cells 18 to expand or contract to render haptic feedback or compensate for uneven terrain features, respectively.

For example, in at least one embodiment, processor 44 receives input information from a VR-based or AR-based environment, including information regarding sizes, shapes, and locations of virtual terrain features, and controls valve 46 to either open or close to let gas into or out of a bladder cell 18 as needed to render the virtual terrain feature.

Range finder 48 may be used to measure the height of a bladder cell 18 to provide feedback information to processor 44 as part of a control loop. Similarly, pressure sensor 50 can be used to measure pressure within the bladder cells 18 and provide such information to processor 44 as part of a control feedback loop.

In at least one embodiment, one or more accelerometers 54 and/or gyroscopes 56 can be used to measure and record acceleration and position information to monitor and track the gait of the user. Additionally, or alternatively, one or more accelerometers 54 and gyroscopes 56 can be used to detect uneven terrain and relay information to processors 44 of one or multiple mechatronic units 40 to expand or contract certain bladder cells 18 in order to compensate for uneven terrain and keep the user's foot level and stable.

One or all of the above-noted components of mechatronic unit 40 may be wired, printed, or otherwise electronically connected with other components via circuit board 42. In at least one embodiment, one or more of the components of mechatronic unit 40 may be disposed separately on or within plate 28 of orthotic device 10. In addition, in embodiments having multiple mechatronic units 40 within plate 28, each mechatronic unit 40, or at least processors 44 thereof, may communicate via a central processing unit 60, as illustrated in FIG. 5B.

FIG. 5B illustrates a schematic view of a plurality of mechatronic units 40a-40e connected together within a plate 28 of an orthotic device 10, according to the present disclosure. In at least one embodiment, the position of each mechatronic unit 40a-e may correspond in position with a bladder cell 18. Central processing unit 60 may be hardwired to each mechatronic unit 40a-e or communicate wirelessly with each mechatronic unit 40a-e. As such, in at least one embodiment, central processing unit 60 is embedded within plate 28. In at least one embodiment, central processing unit 60 may be disposed outside of plate 28 and orthotic device 10 but wirelessly communicate with the various mechatronic units 40a-e. In such an embodiment, each mechatronic unit 40a-e may include a receiver and transmitter to communicate with a receiver and/or transmitter of the central processing unit 60.

Central processing unit 60 may serve a number of functions, including sending instructions to processors 44 of mechatronic units 40a-e, receiving and/or recording information gathered by various components of mechatronic units 40a-e as described above, and/or providing power to the various components of each mechatronic unit 40a-e. In at least one embodiment, central processor 60 serves to relay information from one mechatronic unit 40a-e to another.

For example, in a scenario where an orthotic device 10 of the present disclosure is being used to compensate for uneven terrain, one mechatronic unit 40a-e associated with a bladder cell 18 that contacts a raised terrain feature during a user's step may communicate to another bladder cell 18, such as an adjacent bladder cell 18 or other bladder cell 18, to expand or contract to keep the user's foot level. In at least one embodiment, this communication can be relayed through central processor 60. Alternatively, or additionally, each mechatronic unit 40a-e can be directly connected to each other mechatronic unit 40a-e for communication and/or power supply purposes without the need for central processor 60.

In at least one embodiment, central processing unit 60 includes a power source. In at least one embodiment, a power source is a separate component disposed elsewhere. In at least one embodiment, for example, a power supply is not embedded on or within plate 28, but rather disposed on or within shoe upper 12 or otherwise outside orthotic devices 10 described herein. Preferably, a power source is located in a position where the power source is not likely to be damaged during use. Such a location, for example, may be the back of shoe upper 12 or within plate 28 such that a user is less likely to strike that portion of the user's shoe or orthotic device 10 against a rock, curb, or other terrain feature.

### Valve Assembly & Operation

FIG. 6 illustrates a cross-sectional view of an embodiment of an orthotic device 10, taken along line 6-6 indicated in FIG. 3A, including mechatronic units 40a, 40b integrated into plate 28. Mechatronic units 40a, 40b include valves 46a, 46b, respectively, that interface with bladder cells 18a, 18b, respectively. Mechatronic units 40a, 40b may operate similarly to one another to control respective bladder cells 18a, 18b. Reference will be made here to mechatronic unit 40a configured to control bladder cell 18a, but the description thereof is also relevant to mechatronic unit 40b and associated valve 46b and bladder cell 18b.

A processor, either as part of mechatronic unit 40a or a central processor 60 communicating therewith, controls the opening and closing of valve 46a. The opening and closing of valve 46a allows the selective flow of gas through passageway 62a between port 36a and bladder cell 18a, either in or out of bladder cell 18a. As described above, in at least one embodiment, orthotic device 10 utilizes passive control where the weight of a user pressing downward onto bladder cell 18a forces air out through passageway 62 and port 36 when valve 46 is opened. Gas will not pass out of bladder cell 18a if valve 46a is closed.

If bladder cell 18a is contracted during a user's step (because the valve 36a is opened), valve 36a can remain open during the user's swing motion when orthotic device 10 is not in contact with the ground and the weight of the user is not pressing downward on bladder cell 18a. During this swinging motion, open valve 36a allows air to flow back into bladder cell 18a, the air being drawn back into bladder cell 18a due to sidewalls 38 of bladder structure 30 elastically rebounding to a default/resting configuration.

Valve 36a may not always open for the entirety of a user's stepping motion when the user puts weight on bladder structure 30 such that the valve may only allow bladder cell 18a to partially contract or reduce in volume during a user's step. In this way, valve 36a interfaces with bladder cell 18a and passageway 62a, which leads from bladder cell 18a to port 36a, to control the variable volume of 18a in order to render terrain surfaces or compensate therefore as described herein.

Additionally, or alternatively, mechatronic unit 40a may also include one or more pressurized gas sources and or pumps that communicate with bladder cells 18a, 18b to actively inflate (expand) and/or deflate (contract) individual bladder cells 18a, 18b. In at least one embodiment, such pressurized gas sources and pumps may be integrated with or separate from the mechatronic units 40a, 40b.

In at least one embodiment, for example, a pressurized source of sodium-azide or other deflagrating material or compressed gas can be introduced into bladder cell 18a to inflate (expand) bladder cell 18a as needed. Likewise, a pump may actively pump out gas from bladder cell 18a to deflate (contract) bladder cell 18a as needed. In such an active embodiment, one or more valves 46a may include multi-way valves to a provide passageway for gas to exit through port 36a or a separate passageway in communication with a pressurized gas source as described above. The source of pressurized gas and/or deflagrating material may be stored on a useful location of the orthotic device, such as back of shoe upper 12 or within plate 28 such that a user is less likely to strike that portion of the user's shoe or orthotic device 10 against a rock, curb, or other terrain feature.

FIG. 7A illustrates a perspective view of an embodiment of a valve 36 used in at least one orthotic device 10 described herein. In at least one embodiment, valve 36 includes a housing 64. Along these lines, FIG. 7B illustrates a cross-sectional view of valve 36 illustrated in FIG. 7A, taken along line 7B-7B indicated in FIG. 7A. In at least one embodiment, housing 64 may prevent leakage between the soft polymer of plate 28 and the more rigid valve body 68 during use. Cap 66 prevents inflow of liquid polymer materials of plate 28 into valve body 68 through valve channel 70 during the molding process.

In at least one embodiment, housing 64 includes an extension 72 that forms a recessed channel 74 around valve channel 70. During the molding process when valve 36 is embedded within plate 28 of orthotic devices 10 described herein, liquid polymer material of plate 28 enters into recessed channel 74 and forms a ring around valve channel 70. The interlocking interface between this ring portion of plate 28 within recessed channel 74 of valve housing 64 enables the softer material of plate 28 to flex during repeated use without forming leaks between valve body 68, with associated valve channel 70, and plate 28.

In addition, valve housing 68 may also include cap 66, which temporarily protects opening 76 of valve channel 70 so that liquid polymer material of plate 28 does not enter therein during the molding process. After the plate 28 cures or otherwise solidifies, cap 66 can be broken off or otherwise removed to expose valve channel 70. In at least one embodiment, valve 36 includes more than one valve channel 70, one of which extends between valve body 68 and bladder cell 18 and one which extends between valve body 68 and port 36 as shown in FIGS. 3A and 3B.

FIG. 8 illustrates a perspective view of a mold 76 used to form a plate 38 of an orthotic device 10 as described herein. To form the plates 28 described herein having one or more mechatronic units 40 embedded therein, mechatronic units 40 are first placed in mold 76. In at least one embodiment, mechatronic units 40 are wired via electrical cables 78, such as ribbon cables, and tubes 80 are placed to extend from valves 46 of mechatronic units 40 out the sides of mold 76. Then, liquified polymer material for forming plate 28 are poured into mold 76. Upon curing or otherwise solidifying of the plate 28, mechatronic units 40 are embedded within plate 28 and tubes 80 form passageways 62 and 70 shown in FIGS. 6 and 7B, respectively. After plate 28 is formed, tubes 80 extending outside of mold 76 can be trimmed to form ports 36 shown in FIGS. 3A and 3B.

In at least one embodiment, electrical cable 78 are arranged such that they can bend, expand, contract, or otherwise flex during use of an orthotic device 10. In this way, bladder structure 30 and plate 28 can flex and stretch during use without damaging or disconnecting electrical cables 78 during use.

### Exemplary Use in a VR or AR Environment

FIG. 9 illustrates a user wearing orthotic devices 10 while walking on treadmill 82 within a VR-based environment 84. As described above, orthotic devices 10 of the present disclosure allow a user to receive haptic feedback mimicking terrain features rendered in the VR-based environment 84. In addition, treadmill 82 and/or other VR or AR equipment known in the art can be used in conjunction with orthotic devices 10 to enhance the VR/AR experience. For example, treadmill 82 may be configured to further enhance the variety of haptic feedback made available to the user by providing large slope and other gross terrain features in addition to the fine terrain features and slopes provided by orthotic devices 10.

Spatial information regarding terrain features and relative positions to the user's feet and body can be synced with the various processors 44 of orthotic devices 10 described herein (and/or to one or more central processing units 60 associated with the orthotic devices 10) to determine the position of orthotic devices within VR-based environment 84. Real-time foot tracking can be synchronized with the graphical projections of the VR-based environment 84. Foot-tracking may involve determining that heel-strike is about to occur when the heel (or heel marker being tracked by hardware of the VR-based environment 84) reaches a local minimum. Toe-off may be detected similarly when the toe (or marker being tracked by hardware of the VR-based environment 84) rises above a local minimum.

Determination of foot position within the VR-based environment 84 can trigger corresponding actuation of bladder cells 18 so that the ground features of the VR-based environment 84 are rendered via haptic feedback to the user. For example, when the foot is about to collide with an uneven surface of the VR-based environment 84 (e.g., right before heel strike), commands are sent to the various processors 44 of the mechatronic units 40 of the desired bladder cells 18 (e.g., a particular subset) directing valves 46 to open. The bladder cells 18 with open valves 46 then deflate under foot pressure while other bladder cells 18 with closed valves 46 (or not fully open valves) remain inflated (or deflate to a lesser degree). Thus, the user feels high or low spots under each foot that matches the surface seen in the VR-based environment 84. As the user lifts a foot off the surface, the orthotic device 10 resets and allows bladder cells 18 to re-inflate. As the VR-based environment 84 updates while the user moves therethrough, the corresponding terrain features may be rendered by orthotic devices 10 with each new step.

Accordingly, as the user steps on a virtual terrain feature such as a small rock or sloped object, the orthotic devices 10 described herein actuate and transfer the physical sensation of such terrain features through orthotic devices 10 to the user's feet via haptic feedback. This haptic feedback is thus capable of rendering fine terrain features while the user walks on the flat surface of treadmill 82. The same functionality can be utilized with orthotic devices 10 described herein to provide haptic feedback to a user in an AR-based environment as well, as discussed above.

### Examples

A pilot study was performed to assess the performance of orthotic devices such as those disclosed herein (referred to in this example as “smart shoes” or SS). Participants included a group of PD participants and a group of healthy elderly (HE) participants to compare PD responses against a healthy population. Participants were placed on a treadmill that allowed movement within a VR environment. The VR environment included several paths “paved” with cobblestone. Participants were asked to walk along the path with a comfortable walking speed while wearing their regular walking shoes and the orthotic devices. Three test sessions were conducted, including walking with regular shoes (Reg), fully inflated SS (SS-I), and SS with terrain rendering enabled (SS-R). The SS only rendered uneven cobblestone surfaces when the participant steps onto these surfaces during the SS-R trials.

Three trials with a minimum of ten steps per foot after reaching steady state was captured by the motion capture system when the participant walked on the VR cobblestone walkway. Irregular gait caused by turning in VR was minimized by only recording steps when participants walked in a straight line.

Kinesthetic aspects of the haptic responses were evaluated with spatiotemporal gait properties and kinematics derived from motion capture data to evaluate the change in gait characteristics associated with walking with the different shoe configurations. This allows measurement of kinesthetic haptic response (e.g., changes in motion). Participant questionnaire scores evaluating SS properties, SS performance in VR, and its effect on gait were also collected.

To understand the effects of subject type and shoe configuration on spatiotemporal gait properties and kinematics, we employed a two-way ANOVA with repeated measures: two levels of subject type and three levels of shoe configurations with repeated measures on the shoe configuration. The subject type was either HE or PD while shoe configuration was either Reg, SS-I, or SS-R. Post hoc comparison with Bonferroni correction was used to identify statistical significance between multiple cases. We used a significance level of α=0.05. Bonferroni correction was applied to the p value instead of a such that F and t statistics could be reported without confusion. Likewise, there were three comparisons needed to evaluate differences between shoe configuration which include SS-R/Reg, SS-I/Reg, and SS-R/SS-I; thus, p values were scaled up by a factor of three instead of scaling alpha.

Gait Spatiotemporal Parameters:

Speed, cadence step length and step width were Height Normalized (HN) for each participant. Results of the ANOVA reveal that subject type had a statistically significant effect on HN Speed (F(1, 32)=9.15, p=0.005) and HN Cadence (F(1, 32)=17.69, p<0.001). Further t-test analysis reveals that PD participants were on average 0.08 slower, or about 27% overall, than HE participants (t(27)=3.02, p=0.005). Similarly, PD participants had a significantly slower HN Cadence by an average of 11.0 (t(27)=4.20, p=0.005). These results are expected because PD participants tend to have a slower gait than HE participants [35], which has also been demonstrated on real cobbled surfaces use to generate the VR simulations here.

Shoe configuration had a statistically significant effect on minimum toe clearance (MTC) (F(2, 32)=3.84. p=0.034). Results of the t-test indicate that participants wearing SS-R increased their toe clearance by 2.39 cm when compared to Reg (t(27)=2.66, p=0.039). (MTC) is the most important gait metric associated with the highest risk of unintentional ground contact (e.g., tripping) [36], leading to potential falls. Existing literature reports increased MTC when walking on irregular floor surfaces and while wearing heavier footwear to avoid tripping when stepping over obstacles. The ANOVA and t-test results suggest that increased MTC may be due to the uneven terrain rendering provided by the SS-R.

Gait Kinetic Parameters:

Gait kinematic parameters, including hip angle, knee angle, and ankle angle in the sagittal plane are important measures of how people walk. In this work, kinematic angles are normalized for each step from heel-strike to the subsequent heel-strike on the same foot, or 0% to 100% gait cycle, within groups of HE participants and participants with PD.

Two-way ANOVA was performed on the maximum and minimum angles of hip, knee, and ankle, as well as the range of motion for each respective joint. Results reveal that subject type had a statistically significant effect on maximum knee angle (F(1, 27)=5.67, p=0.025). Further t-tests reveal that PD patients compared to HE had reduced maximum knee angle of 6.5° (t(27)=2.38, p=0.024). This is likely caused by typical PD shuffle gait.

Shoe configuration had statistically significant effects on minimum knee angle (F(2, 27)=5.07, p=0.014) and maximum ankle angle (F(2, 27)=7.79, p=0.002). Participants that walked with SS-R showed minimum knee angle reduced by 16.9° compared to Reg (t(27)=2.88, p=0.023) and a 16.0° reduction for SS-I compared to Reg (t(27)=2.61, p=0.043). Both HE and PD also show statistically significant increases in maximum ankle angle (dorsiflexion) during the swing phase; SS-R increased by an average of 6.5° compared to Reg (t(27)=3.29, p=0.008); SS-I increased by 7.0° compared to Reg (t(27)=3.53, p=0.005). These increases in knee flexion and ankle dorsiflexion are likely the direct cause of the increased MTC [39] noted in the last section attributed to walking over irregular surfaces.

Two-way ANOVA was performed on ankle angle during the four different stages of the gait, through the heel rocker and the ankle rocker (i.e., 5%, 10%, 15% (about footflat) and 25% (about mid-stance)). These four stages were selected since they indicate the initial SS deflation process to the full deflation at about mid-stance.

Results indicate that subject type had a statistically significant effect on ankle angle at 5% gait cycle (F(1, 32)=4.26, p=0.049). T-tests indicate that PD participants held a statistically significant higher ankle angle on average when compared to HE (t(27)=2.06, p=0.049); PD participants held an average ankle angle of 83.4° while HE participants held an average ankle angle of 78.9°. This was expected as people with PD tend to have reduced ankle flexion with shuffle gait.

Shoe configuration had a statistically significant effect on ankle angle at 15% gait cycle (F(1, 32)=4.65, p=0.018) and 25% gait cycle (F(1, 32)=3.94, p=0.031). At 15% gait cycle, participants who used SS-R increased their ankle angle relative to Reg by 8.1° (t(27)=2.87, p=0.024) and by 6.1° for 25% gait cycle (t(27)=2.60, p=0.044). There was no statistical significance between Reg and SS-I for 15% and 25% gait cycle, which suggests that rendering provided by SS-R was important.

Questionnaire:

A questionnaire was administered immediately after each session to collect subjective participant feedback data before progressing with the next shoe configuration. The participant was asked to answer eight questions regarding their VR and shoe experiences: Q1: realism of graphics, Q2: realism of walking on cobblestone, Q3: walking difficulty, Q4: walking difficulty on cobblestone, Q5: shoe stability, Q6: likelihood of ankle roll over, Q7: fear or walking, and Q8: max comfortable walking speed.

Results of the ANOVA show that shoe configuration had a significant effect on Q2 “Realism of walking on Cobblestone” (F(2, 32)=8.91, p=0.001). When using the SS-R, participants reported a significant 3.78 point increase in realism when walking on cobblestone compared to Reg (t(27)=4.14, p<0.001). Similarly, SS-R was reported to be on average 2.52 points more realistic than SS-I (t(27)=2.76, p=0.031). This suggests that shoe compliance (i.e., SSI) was a factor for increased realism on cobblestone, but SSR rendering played an even bigger role.

Shoe configuration was also statistically significant in Q3 “Walking Difficulty” (F(2, 32)=8.62, p=0.001). There was a significant 3.53 point increase in walking difficulty, when comparing SS-R to Reg (t(27)=3.61, p=0.004) and a 3.51 point increase when comparing SS-I to Reg (t(27)=3.58, p=0.004). As expected, walking with the SS (SS-I and SS-R) is reported to be more difficult than with Reg, which could be related to shoe weight and compliance.

Shoe configuration was also statistically significant in Q4 “Walking Difficulty on Cobblestone” (F(2, 32)=8.19, p=0.002). SS-R was 3.84 points more difficult compared to Reg (t(27)=4.04, p=0.001). There was no statistically significant difference in difficulty between SS-I and Reg (t(27)=1.86, p=0.22) nor SS-R and SS-I (t(27)=2.18, p=0.114). These results suggest that walking with the SS-R on cobblestone in notably more challenging than with SS-I or Reg.

Summary:

There were significant differences correlated to shoe configuration, specifically the SS-R, for MTC, minimum knee angle, max ankle angle, and ankle angle at 15% and 25% gait cycle. These are expected with gait changes associated with walking on irregular surfaces. Lastly, we hypothesized that both groups would subjectively rate a higher VR experience using the SS-R vs SS-I or Reg. According to the questionnaire, SS-R was rated as the most realistic followed by SS-I and Reg. Users also reported significantly increased difficulty walking on cobblestone with the SS-R, which is a goal for making the terrain enabled VR experience more realistic.

Two tailed t-tests were used to test for statistical significance of height normalized speed and cadence between this study and those reported in a previous study comparing walking parameters on flat ground and real cobblestone. With the exception of HE cadence (t(12)=2.61, p=0.023), the results suggest that there are no statistically significant differences between the results in this study and those derived from the previous study. This suggests that participants using the proposed terrain rendering system selected similar normalized speed and cadence as HE and PD participants on actual cobble, which is a good indicator of the realism created by the proposed system.

All of these results suggest that SS-R terrain rendering coupled with the VR system provided enhanced VR experiences typified by objective measures of gait variations also supported by subjective questionnaire results. The aforementioned increases in realism could be leveraged in PD subject rehabilitation, for example. The goal of such rehabilitation would be to provide challenges that increase ankle dorsiflexion during heel rocker and MTC, which are related to gait compensatory mechanisms that are important for participants to regain desired balance and motor functions, especially among the PD population.

### CONCLUSION

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

We claim:

1. An orthotic device for haptic terrain feedback and control, comprising:
a plate comprising a mechatronic unit;
a bladder structure secured to the plate, the bladder structure comprising a bladder cell; and
a passageway extending from the bladder cell to an exterior of the orthotic device in communication with ambient air,
wherein the mechatronic unit is operatively coupled to the bladder cell so as to control inflation and deflation of the bladder cell via movement of gas through the passageway.

2. The orthotic device of claim 1, wherein the plate comprises a plurality of mechatronic units and a plurality of bladder cells, each mechatronic unit associated with a corresponding bladder cell.

3. The orthotic device of claim 1, wherein the mechatronic unit comprises a valve moveable between an open position and a closed position to control movement of gas through the passageway.

4. The orthotic device of claim 1, wherein the bladder cell is at least partially defined by one or more sidewalls.

5. The orthotic device of claim 1, wherein the bladder cell is at least partially defined by the plate.

6. The orthotic device of claim 1, wherein the bladder structure comprises at least four bladder cells.

7. The orthotic device of claim 2, wherein the plurality of bladder cells are at least partially defined by separate sidewalls such that the bladder cells are independent of one another.

8. The orthotic device of claim 7, wherein at least one of the separate sidewalls is curvilinear and omits T-junctions.

9. The orthotic device of claim 1, wherein the bladder structure comprises a composite material.

10. The orthotic device of claim 9, wherein the composite polymer material comprises a fabric-polymer composite material.

11. An orthotic device for haptic terrain feedback and control, comprising:
a bladder structure comprising a plurality of bladder cells; and
a mechatronic plate having one or more mechatronic units disposed therein, each mechatronic unit of the one or more mechatronic units being associated with a corresponding bladder cell of the bladder structure and each mechatronic unit comprising a valve,
wherein the valves are configured to be selectively opened and closed to allow a gas to flow in and out of corresponding bladder cells independently.

12. The orthotic device of claim 11, wherein each of the one or more mechatronic units comprises a processor configured to control the valve.

13. The orthotic device of claim 12, wherein each of the one or more mechatronic units further comprises: an accelerometer; a range finder; and a pressure sensor.

14. The orthotic device of claim 11, wherein each of the plurality of bladder cells is at least partially defined by a sidewall.

15. The orthotic device of claim 14, wherein none of each of the plurality of bladder cells shares a common sidewall such that each of the plurality of bladder cells is separate from the others.

16. The orthotic device of claim 11, the valve comprising:
a valve channel extending from a valve body; and
a housing surrounding a valve body, the housing comprising;
an extension extending from the housing and forming a recessed channel, the recessed channel forming a ring around the valve channel.

17. The orthotic device of claim 16, wherein the valve is embedded within the mechatronic plate such that a portion of the mechatronic plate extends into the recessed channel to form a tight seal between the mechatronic plate and the valve.

18. An orthotic device for terrain control, comprising:
a bladder structure that includes
a plurality of bladder cells; and
one or more sidewalls at least partially defining the plurality of bladder cells; and
a plate secured to the bladder structure, the plate comprising:
a mechatronic unit operatively coupled to the bladder structure to enable controllable inflation and deflation of one or more of the bladder cells;
a port; and
a passageway extending through the plate between at least one of the plurality of bladder cells and the port.

19. The orthotic device of claim 18, wherein the plate comprises a valve configured to selectively open and close the passageway.

20. The orthotic device of claim 18, wherein the plate comprises a plurality of mechatronic units, each associated with a respective bladder cell.
